(19) United States
(12) Patent Application Publication
(10) Pub. No.: US 2010/0297675 A1
(43) Pub. Date: Nov. 25, 2010

(54) SUBSTRATE AND METHOD FOR CULTURING BREAST CELLS

(76) Inventors: Huayun Deng, Mayfield Heights, OH (US); Joydeep Lahiri, Painted Post, NY (US); Hui Su, Big Flats, NY (US)

Correspondence Address: CORNING INCORPORATED, SP-TI-3-1, CORNING, NY, 14831, US

(21) Appl. No.: 12/623,884

(22) Filed: Nov. 23, 2009

Related U.S. Application Data
(60) Provisional application No. 61/117,366, filed on Nov. 24, 2008.

(51) Int. Cl. G01N 33/574; C12N 5/07

(52) U.S. Cl. 435/7.23; 435/395

(57) ABSTRACT

A cell culture article includes a porous substrate having a plurality of pores and a plurality of interstices in communication with the pores. At least some of the plurality of pores and interstices are sufficiently large for two or more mammary epithelial cells to cluster within the pores or interstices. Non-malignant mammary epithelial cells or breast cancer cells may not attach strongly to the substrate surface, which may encourage cell-cell interaction. In many cases, the article is desirably free of components of unknown origin. The articles may be capable of maintaining culture of malignant and non-malignant mammary epithelial cells and allowing for development of in vivo-like morphologies or characteristics of such cells.

### RELATED APPLICATION

This application claims the benefit of priority to U.S. Provisional Patent Application Ser. No. 61/117,366, filed on Nov. 24, 2008, which application is hereby incorporated by reference in its entirety to the extent that it does not conflict with the present disclosure.

### FIELD

The present disclosure relates to cell culture, and more particularly to culture of breast cells and to substrates that promote in-vivo like characteristics of cultured breast cells.

### BACKGROUND

In vitro studies of human cancer including breast cancer have been primarily carried out with established cell lines on two-dimensional (2D) cell culture surfaces. However, many cells, such as non-malignant and malignant mammary cells and other differentiated cell types, rapidly lose their specific morphology and cellular functions when cultured on 2D surfaces. To overcome this deficiency of 2D surfaces, extracellular matrices (ECM) have been employed to establish and maintain functional specificity of cells, such as mammary cells, in cell culture. The ECM materials provide a three-dimensional (3D) substrate for culturing cells that more closely mimic in vivo environments and promote in vivo-like morphology of cultured cells. For example, non-malignant breast cells cultured on substrates having ECM material, such as Matrigel™ (BD Biosciences—Discovery Labware, Inc.), have been shown to organize into polarized and growth-arrested colonies with characteristic morphological features of mammary acini, while they form monolayers instead of the acini structures on 2D culture surfaces. Unlike non-malignant breast cells, malignant cells develop into colonies of different morphologies with some common features such as disorganized nuclei structure, failure to arrest growth and loss of tissue polarity.

In addition to morphological differences between breast cells cultured in 2D and 3D, differences in gene expression, signal transduction pathways and apoptotic sensitivity in response to chemotherapeutic agents has been observed. For example, breast cancer cells cultured on Matrigel™—based 3D substrates have been shown to express key molecular targets such as betal-integrin and TACE/ADAM17. While Matrigel™—based 3D cell culture has successfully demonstrated the significance of 3D culture in cancer research; it has some distinct disadvantages that significantly limit its applications beyond research labs. For example, as Matrigel™ is an animal-origin extracellular matrix, it is inconsistent in composition which results in inconsistent culture results. In addition, it includes various proteins and enzymes which can interfere with various cellular assays. Further, its gel format makes automatic handling difficult.

### BRIEF SUMMARY

Among other things, the present disclosure describes cell culture articles having chemically-defined porous substrates that support robust culture of malignant and non-malignant mammary epithelial cells that exhibit in vivo like morphology, without the use of chemically-undefined ECM materials such as Matrigel™. The porous substrates provide a three-dimensional scaffold that is believed to promote the in vivo like morphology or characteristics of the cells in a reproducible manner.

In various embodiments, a cell culture article includes a porous substrate having a plurality of pores and a plurality of interstices in communication with the pores. At least some of the plurality of pores and interstices are sufficiently large for two or more mammary epithelial cells to cluster within the pores or interstices. Non-malignant mammary epithelial cells or breast cancer cells do not adhere to the substrate, which may encourage cell-cell interaction. In many cases, the article is free of components of unknown origin.

Embodiments of cell culture articles are shown herein to support culture of mammary epithelial cells having in vivo-like morphology or characteristics, such as formation of actini structures in non-malignant mammary epithelial cells, formation of mass cell structures with robust cell-cell interaction and disorganized nuclei in non-invasive breast cancer cells, formation of elongated cell bodies resembling invasive processes in invasive malignant breast cancer cells, response to anti-cancer agents by breast cancer cells, and reversion of malignant phenotype of breast cancer cells. Such culture articles may be useful in screening candidate agents for treating breast cancer.

One or more of the various embodiments presented herein provide one or more advantages over prior articles and systems for culturing mammary epithelial cells. For example, unlike substrates employing animal-derived ECM materials, the porous substrates described herein are readily tunable and reproducible, and may provide more consistent cell culture results. Further, the substrates provide a solid scaffold that can support easy sterilization, handling and adaptation to automation. These and other advantages will be readily understood from the following detailed descriptions when read in conjunction with the accompanying drawings.

### BRIEF DESCRIPTION OF THE DRAWINGS

FIG. 1 is a schematic drawing showing an embodiment of a method for forming a porous substrate for culturing cells.

FIG. 2 is a schematic cross-section of an embodiment of a cell culture article having a porous substrate for culturing cells.

FIG. 3 is a schematic cross section of an embodiment of a portion of a porous substrate with cell clusters in the pores and interstices of the substrate.

FIGS. 4-7 are flow diagrams of embodiments of methods for screening candidate compounds or agents employing a cell culture article having a porous substrate as described herein.

FIGS. 8A-B are confocal fluorescence images of non-malignant mammary epithelial cells cultured on a cell culture article having a porous polydimethylsiloxane (PDMS) substrate, with FIG. 8B being at higher magnification.

FIG. 9 is a confocal fluorescence image of malignant breast cultured on a cell culture article having a porous PDMS substrate.

FIG. 10 is a confocal fluorescence image of invasive malignant breast cultured on a cell culture article having a porous PDMS substrate.

FIGS. 11A-C are confocal fluorescence images of malignant breast cancer cells cultured on a two-dimensional TCT substrate without treatment (A), with treatment with MAPK inhibitor, PD98059 (B), and with treatment of PI3K inhibitor, LY294002 (C).

FIGS. 12A-C are confocal fluorescence images of malignant breast cancer cells cultured on a porous PDMS substrate without treatment (A), with treatment with MAPK inhibitor, PD98059 (B), and with treatment of PI3K inhibitor, LY294002 (C).

FIGS. 13A-C are confocal fluorescence images of malignant breast cancer cells cultured on a porous PDMS substrate without treatment (A), with treatment with MAPK inhibitor, PD98059 (B), and with treatment of PI3K inhibitor, LY294002 (C).

The schematic drawings presented herein are not necessarily to scale. Like numbers used in the figures refer to like components, steps and the like. However, it will be understood that the use of a number to refer to a component in a given figure is not intended to limit the component in another figure labeled with the same number. In addition, the use of different numbers to refer to components is not intended to indicate that the different numbered components cannot be the same or similar.

### DETAILED DESCRIPTION

In the following detailed description, reference is made to the accompanying drawings that form a part hereof, and in which are shown by way of illustration several specific embodiments of devices, systems and methods. It is to be understood that other embodiments are contemplated and may be made without departing from the scope or spirit of the present disclosure. The following detailed description, therefore, is not to be taken in a limiting sense.

1. Definitions

All scientific and technical terms used herein have meanings commonly used in the art unless otherwise specified. The definitions provided herein are to facilitate understanding of certain terms used frequently herein and are not meant to limit the scope of the present disclosure.

As used herein, “pore” means a cavity or void in a surface, a body, or both a surface and a body of a solid article, where the cavity or void has at least one outer opening at a surface of the article.

As used herein, “interstice” means a cavity or void in a body of a solid polymer not having a direct outer opening at a surface of the article, i.e., not a pore, but may have an indirect outer opening or pathway to an outer surface of the article by way of one or more links or connections to adjacent or neighbor “pores” “interstices,” or a combination thereof.

As used herein, “porous network” means the combined or total void-volume, consisting of the pores and the interstices of an article.

As used herein, “porosity” means the ratio of the volume of a porous network of a material to the volume of the material's mass.

As used herein, “continuous void phase” refers to an article having an interconnected porous network that is substantially free of “dead ends” or “no-outlets” such as having only a single connection to another interstice, or “isolated voids,” that is, interstices having no interconnectivity.

A “semi-continuous void phase” refers to an article having an interconnected porous network that may have some amount of the above mentioned “dead ends” or “isolated voids,” such as from about 1 to about 20% by volume.

As used herein, “substrate”, as it relates to a cell culture substrate, refers to a material in or on which cells may be cultured. For example, a porous substrate may have voids in which cells may be cultured. “Cell culture” or “cell culturing” refers to the process by which either prokaryotic or eukaryotic cells are grown under controlled conditions, and may include the culturing of complex tissues, organs, or cell systems.

As used herein, “optical density”, as it relates to a porous substrate material, means a measure of the transmittance of a given wavelength of light through given length, depth or thickness of the porous substrate material. Optical density measurements may be performed in the presence or absence of culture media.

As used herein, “retention rate”, as it relates to cells and a culture surface, means the percentage of viable cells that are retained, after a period of time, on the surface following gentle washing with cell culture medium, phosphate buffered saline, or other suitable solution. Gentle washing may include, for example, shaking the cell culture article at about 2000 rpm for about 15 seconds. As used herein, cells are considered “non-attachable” to the surface or substrate, or are considered to “not attach” to the surface or substrate, if the number of cells that attach to the surface or substrate is 50% or less than the number of cells that attach to a TCT surface (tissue culture treated polystyrene). To compare the ability of cells to adhere to a three-dimensional substrate material, such as a porous polymer, a non-porous flat two-dimensional surface may be formed from the material and compared to a TCT surface for determining whether the cells are “non-attachable” to the surface. In some embodiments, the number of cells that attach to a non-attachable surface or substrate material is 25% or less, 15% or less, or 10% or less than the number of cells that attach to a TCT surface of the like size under like conditions.

As used herein, “assay,” “assaying” or like terms refers to an analysis to determine, for example, the presence, absence, quantity, extent, kinetics, dynamics, or type of a cell's growth characteristics or response to an exogenous stimuli, such as a candidate compound, culture media, substrate coating, or like considerations.

Unless stated to the contrary, reference herein to relative percents or percentages of components of a composition are by weight.

“About” modifying, for example, the quantity of an ingredient in a composition, concentrations, volumes, process temperature, process time, yields, flow rates, pressures, and like values, and ranges thereof, employed in describing embodiments of the disclosure, refers to, for example, variation in the numerical quantity that can occur, for example, through typical measuring and handling procedures used for making compounds, compositions, concentrates or use formulations; through inadvertent error in these procedures; through differences in the manufacture, source, or purity of starting materials or ingredients used to carry out the methods; and like considerations. The term “about” also encompasses amounts that differ due to aging of, for example, a composition, formulation, or cell culture with a particular initial concentration or mixture, and amounts that differ due to mixing or processing a composition or formulation with a particular initial concentration or mixture. Whether modified by the term “about” the claims appended hereto include equivalents to these quantities.

“Optional,” “optionally,” or like terms refer to the subsequently described event or circumstance can or cannot occur, and that the description includes instances where the event or circumstance occurs and instances where it does not. For example, the phrase “optional component” means that the component can or can not be present and that the disclosure includes both embodiments including and excluding the component.

As used in this specification and the appended claims, the singular forms “a”, “an”, and “the” encompass embodiments having plural referents, unless the content clearly dictates otherwise. As used in this specification and the appended claims, the term “or” is generally employed in its sense including “and/or” unless the content clearly dictates otherwise.

As used herein, “have”, “having”, “include”, “including”, “comprise”, “comprising” or the like are used in their open ended sense, and generally mean “including, but not limited to”. It will be understood that “consisting essentially of”, “consisting of” and the like are subsumed in “comprising” and the like. Accordingly, a porous substrate comprising polydimethylsiloxane (PDMS) includes a porous substrate consisting essentially of, or consisting of, PDMS.

“Consisting essentially of”, as it relates to a compositions, articles, systems, apparatuses or methods, means that the compositions, articles, systems, apparatuses or methods include only the recited components or steps of the compositions, articles, systems, apparatuses or methods and, optionally, other components or steps that do not materially affect the basic and novel properties of the compositions, articles, systems, apparatuses or methods. By way of example, items that may materially affect the basic properties of the components of a cell culture article or porous substrate described herein are those components that may impart undesirable characteristics to such an article or substrate. For example, if the article or substrate is clearly intended to closely optically match with cell culture media, a component that results in an undesirable optical mismatch between the cell culture article or substrate and the liquid culture media may be considered to materially affect the basic and novel properties of the article or substrate. An example of a component that may result in an undesirable optical mismatch is an optically opaque entrapped pore-former particles that cannot be substantially removed from a polymer material.

Specific and preferred values disclosed for components, ingredients, additives, cell types, pathogens, and like aspects, and ranges thereof, are for illustration only; they do not exclude other defined values or other values within defined ranges. The compositions, apparatuses, systems and methods of the disclosure include those having any value or any combination of the values, specific values, more specific values, and preferred values described herein.

The use of headers herein is not intended to be limiting. For example, relevant discussion of a property, characteristic, component or the like of a porous substrate may be provided under the heading “cell culture” rather than under the heading of “porous substrate”. One or more embodiments of porous substrates described herein may include such a property, characteristic, component or the like, even though such discussion is not provided under the heading “porous substrate.”

2. Porous substrate

The present disclosure describes, inter alia, cell culture articles having chemically-defined porous substrates that support robust culture of malignant and non-malignant mammary epithelial cells that exhibit in vivo like morphology, without the use of chemically-undefined ECM materials such as Matrigel™. In some embodiments described herein, a porous substrate culture system provides a cell culture environment that allows for phenotypic discrimination between nonmalignant and malignant mammary cells, as the former form polarized, growth-arrested acinus-like colonies whereas the latter form disorganized, proliferative and nonpolar colonies. The system also allows for phenotypic discrimination between non-invasive malignant cells and invasive malignant mammary cells, as the latter fanned a stellate structure of disorganized nuclei and elongated cell body with invasive processes. In various embodiments, the system also allows for phenotypic and functional reversion of malignant mammary cells to non-malignant cells exhibiting growth-arrested acini. Accordingly, the cell culture articles and systems described herein may be of value in screening potential therapeutic agents for treatment of breast cancer.

A substrate capable of providing desirable culture of mammalian epithelial cells includes a plurality of pores and a plurality of interstices in communication with the pores. At least some of the pores and interstices are sufficiently large, i.e. occupy a sufficiently large volume, to allow the mammary epithelial cells to cluster within the pores and interstices. The pores are large enough to allow two or more cells to cluster or 10 or 20 or 50 or 100 or more cells to cluster. However, if the pores and interstices are too large, they may not provide the special constraint to facilitate cell clustering or cell-cell interaction.

In some embodiments, to the surface of the porous substrate material does not encourage cell attachment. While not intending to be bound by theory, it is believed that a non-attachable substrate will encourage the cells to interact with each other rather than with the substrate material. Again, while not intending to be bound by theory, it may be desirable, in some embodiments, for the cells to not only be non-attachable to the surface of substrate material but also to be repelled from the surface of substrate material to further encourage cell-cell interaction.

For example, cells may be further encouraged to interact cell-to-cell if the substrate material is hydrophobic, or has a contact angle of greater than about 30 degrees or greater than about 60; e.g. from about 30 degrees to about 130 degrees, from about 60 degrees to about 130 degrees, or from about 80 degrees to about 130 degrees. If the contact angle is too high (e.g., greater than about 150) or the surface too hydrophobic, transportation of water and nutrients into the pores may be inhibited and compromise the ability to sustain cell growth. By way of example, the Examples presented below show that porous polydimethylsiloxane (PDMS) provides an environment that allows for cell-cell interaction and clustering in the pores and interstices of the PDMS substrate. Examples of other hydrophobic polymers that may readily be employed include polyurethanes, poly(tetrafluoroethylene), poly(methyl methacrylate), poly(vinyl chloride), polyethylene and polypropylene.

Of course, in some embodiments, the cells are attachable to the surface of the porous substrate material. In some embodiments, the porous substrate is hydrophilic (e.g., contact angle of less than about 60 degrees). For example, the relative hydrophobicity or hydrophilicity of the substrate may not have a significant effect on cultured malignant cells (e.g., invasive or non-invasive breast cancer cells), but may affect in-vivo like characteristics of non-malignant mammary epithelial cells. Interaction of non-malignant cells in-vivo often dictates cellular morphology and characteristics, where malignant cells often are not constrained by such cell-cell interaction. For example, non-malignant epithelial cells can form acini structures in-vivo due to cell-cell interaction, which structures may be encouraged in cell culture by hydrophobic three-dimensional substrates. On the other hand, breast cancer cells in-vivo or in-vitro do not form such acini structures. Thus, malignant cells may exhibit in-vivo like morphology or characteristics when cultured on hydrophilic or hydrophobic three-dimensional substrates, while non-malignant mammary epithelial cells may be encouraged to exhibit in-vivo like morphology when cultured on more hydrophobic three-dimensional substrates.

In many embodiments, the cell culture article or the substrate is free of components of unknown composition. The precise composition of some cell culture systems is not known. For example, cell culture systems that employ extracellular matrix (ECM) material, particularly non-synthetic, animal-derived ECM material such as Matrigel™, may include unknown components and may contain unknown percentages of known components. Accordingly, cell culture systems employing such materials may result in varying results due to the non-reproducible nature of the material used. It will be understood that cell culture articles or substrates that are “free of components of unknown composition” may have some de minimis amount of unknown material, but this de minimis amount of material will not adversely affect the reproducibility of manufacture or culture results. For example, some reagents used for making a porous polymer substrate may not be completely pure (e.g., 90%, 95%, 98%, 99%, or 99.5% pure). Yet, polymer substrates made from such reagents will be considered to be free of components of unknown composition.

The porous substrates described herein may provide, in various embodiments, one or more of the following advantages, alone or in combination. Cultured cells can freely migrate, communicate, or contact one another through the interconnected porous structure of the article. Cultured cells can grow into spheroids of certain well defined sizes within each interstice or pore in the porous article. The size of the spheroids can be controlled by defining the interstice or pore distribution of the substrates by judicious selection of the pore-former materials or methodologies. In some embodiments, porous articles have two or more particle size distributions of the interstices of the porous network, which can enhance the communication among cells and can enhance nutrient penetration into and waste transport out-of the interconnected channels of the network within the porous article. In such embodiments, the porous articles having larger interstice and pore sizes are designed to accommodate the cell or cell body growth, and the smaller interstice and pore sizes can be designed to enhance cell communication, nutrient exchange, and waste exchange. The porous articles are, for example, porous solids or porous gels, which can be easily combined-with and separated-from culture media including convenient continuous or semi-continuous culture media exchange.

Porous substrates may be formed from any suitable material or materials. Preferably, the substrate is formed from materials compatible with cells to be cultured and culture media. In numerous embodiments the porous substrate is a polymer. Examples of suitable polymeric materials for cell culture include polyamides, polycarbonates, polyalkylenes, polyalkylene glycols, polyalkylene oxides, polyalkylene terephthalates, polyvinyl alcohols, polyvinyl ethers, polyvinyl esters, polyvinyl halides, polyvinylpyrrolidone, polyglycolides, polysiloxanes, polyurethanes, and copolymers thereof, nitro celluloses, polymers of acrylic and methacrylic esters, hydroxypropyl cellulose, hydroxy-propyl methyl cellulose, hydroxybutyl methyl cellulose, cellulose acetate, cellulose propionate, cellulose acetate butyrate, cellulose acetate phthalate, carboxylethyl cellulose, cellulose triacetate, cellulose sulphate sodium salt, poly(methylmethacrylate), poly(ethyhnethacrylate), poly(butylmethacrylate), poly(isobutylmethacrylate), poly(hexlmethacrylate), poly(isodecylmethacrylate), poly(laurylmethacrylate), poly(phenylmethacrylate), poly(methacrylate), poly(isopropacrylate), poly(isobutacrylate), poly(octadecacrylate), polyethylene, polypropylene poly(ethylene glycol), poly(ethylene oxide), poly(ethylene terephthalate), poly(vinyl alcohols), poly(vinyl acetate), poly vinyl chloride, polystyrene, polyhyaluronic acids, casein, gelatin, gluten, polyanhydrides, polyacrylic acid, alginate, chitosan, and any copolymers thereof, or any combination thereof. In various embodiments, a porous polymer is formed from one or more monomer selected from a siloxane, a vinyl substituted trialkoxy silane, an alpha-olefin, a vinyl ester, an acrylate, an acrylamide, an unsaturated ketone, a monovinylidene aromatic hydrocarbons, and like polymerizable monomers, or a combination thereof. Examples of suitable monomers which can be polymerized or copolymerized to form the articles as disclosed herein include the monovinylidene aromatic hydrocarbons (e.g., styrene, aralkylstyrene, such as the o-, m- and p-methylstyrenes, 2,4-dimethylstyrene, the Ar-ethylstyrenes, p-butylstyrene, and like monomers; and alpha-alkylstyrene, such as alpha-methylstyrene, alpha-ethylstyrene, alpha-methyl-p-methylstyrene, and like monomers; vinylnaphthalene, and like monomers); Ar-halo-monovinylidene aromatic hydrocarbons (e.g., o-, m- and p-chlorostyrenes, 2,4-dibromostyrene, 2-methyl-4-chlorostyrene, and like monomers); acrylonitrile, methacrylonitrile, alkyl acrylates (e.g., methylacrylate, butyl acrylate, ethylhexyl acrylate, and like monomers), the corresponding alkyl methacrylates, acrylamides, (e.g., acrylamide, methylacrylamide, N-butylacrylamide, and like monomers); unsaturated ketones (e.g., vinyl methyl ketone, methyl isopropenyl ketone, and like monomers); alpha-olefins (e.g., ethylene, propylene, and like monomers); vinyl esters (e.g., vinyl acetate, vinyl stearate, and like monomers); vinyl and vinylidene halides (e.g., the vinyl and vinylidene chlorides, and bromides, and like monomers); a vinyl substituted silane such as a vinyl substituted trialkoxy silane, and like monomers, or combinations thereof. In various embodiments, porous polymers are formed from one or more suitable oligomer or one or more oligomerr and one or more momomer. Suitable oligomers include those formed from the monomers described above.

A polymer may be made porous via any suitable mechanism, such as mixing with gas, foaming, use of a pore-forming agent, or the like, prior to or during polymerization. Examples of pore-forming agents that may be used include particles of a simple sugar, a polysaccharide, a polyalkylene glycol, a polyvinylalcohol, ice, a wax, a sublimable material such as solid CO₂, a substance having a melting point lower than that of the polymer formed, a water soluble polymer, a water-insoluble polymer, or a copolymer thereof, a microcapsule having a shell and core where, for example, the shell comprises a monomer insoluble material and the core comprises a water miscible or water soluble material, a micro-balloon having a soluble shell and hollow or gas filled core, or combinations thereof. After a resulting polymerized solid matrix is formed, the pore-forming agent is removed leaving voids in the polymeric matrix. The pore-forming agent may be removed by, for example, contacting the solid polymeric material with a substance capable of dissolving the pore-forming agent, heating the matrix to liquefy the particulate phase, or a combination thereof. Examples of substances that may be used to dissolve the particulate phase include an aqueous liquid; an organic liquid; a supercritical fluid such as CO₂; a low melting solid such as a wax, water or the like; a gas such as air, nitrogen, argon or the like; or a combination thereof.

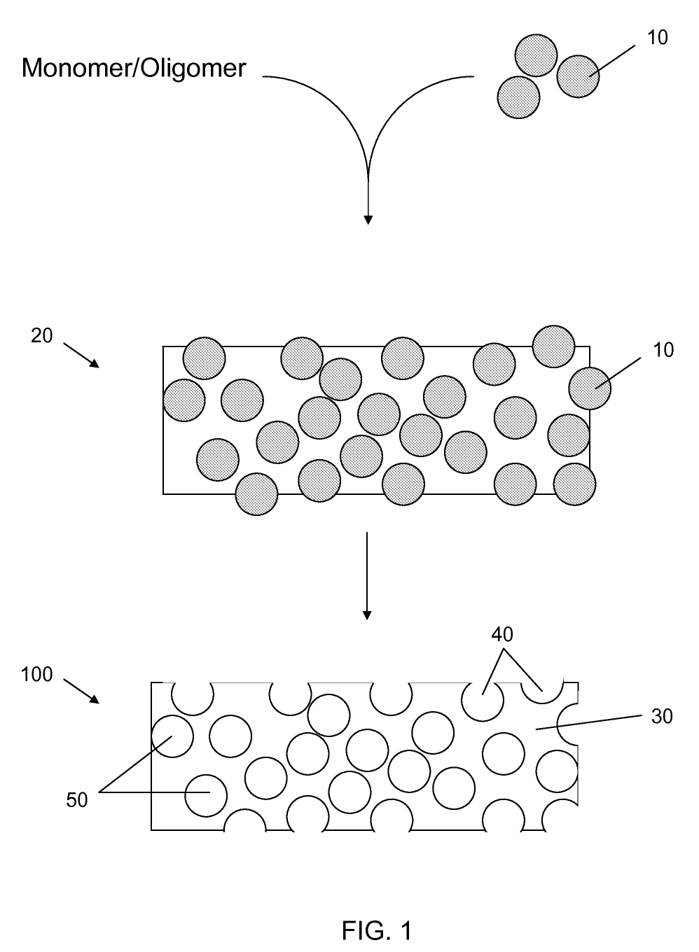

In various embodiments and as depicted in FIG. 1, a porous substrate 100 is prepared by mixing at least one monomer or oligomer for polymerization and at least one particulate pore-former 10, polymerizing the monomers or oligomers to form a particulate filled polymeric matrix 20, and removing the particulate phase. The resulting porous substrate 100 includes a polymeric matrix 30, pores 40 and interstices 50 left by the removed pore-former particles. The particulate phase in the polymer matrix can be, for example, a continuous phase, a semi-continuous phase, a discontinuous phase, or a combination thereof. The momoners or oligomers and particulate pore-former may be mixed in any suitable manner, such as high speed liquid-solid mixing, liquid-solid blending, liquid-solid centrifugation, or a combination thereof. The pore-former packing may be selected based on a particle size ensemble having a void volume that becomes the continuous polymer matrix and the volume-fraction occupied by the particulate pore-former that becomes the void-volume, i.e., the combined interstice and pore-volume, in the resulting cell culture article.

A pore-forming agent may have any suitable particle size to allow mammary epithelial cells to cluster within the pores. In various embodiments, pore-forming agents having a particle diameter of between about 75 micrometers and about 600 micrometers are used to create the pores and interstices of the porous substrate. Such particles sizes provide for pores and interstices of a suitable volume to allow mammary epithelial cells to cluster in the pores and interstices, but are not too large to not encourage cell-cell interaction. In some embodiments, pore-forming agents having a particle diameter of between about 75 micrometers and about 1000 micrometers, between about 100 micrometers and 350 micrometers, or about 200 to about 250 micrometers are used to create the pores and interstices of the porous substrate.

In some embodiments, a second particulate pore former having a smaller diameter is used, in addition to the larger particulate pore-former as described above. The second smaller diameter pore former can be designed to enhance cell communication, nutrient exchange, or waste exchange. The second particulate pore-former may have any suitable size. For example, the second pore-former may have a particle diameter of from about 0.1 micrometers to about 75 micrometers.

In embodiments, having a first particle mixture and the second particle mixture, the respective mixtures can be independently selected from, for example, mono-modal particles, bimodal particles, mono-disperse particles, bi-disperse particles, poly-disperse particles, and a combination thereof. In embodiments, the first particle mixture and the second particle mixture can be comprised of a same substance, or a different substance, yet having different particle size properties, particle size distribution properties, or a combination thereof. In general, as pore-former content increases, porosity and pore size increase, for example, in a linear fashion

A porous polymer as described herein may have any suitable surface area. For example, the porous polymer article may have a surface area of between about 0.1 to about 20 m²/g.

A porous polymer as described herein may have any suitable porosity. For example, the porous polymer may have a porosity of between about 50% and about 95% as measured by mercury or nitrogen porosimetry.

A porous polymer as described herein may have any suitable density. For example, the porous polymer may have a density of between about 1 and about 1000 kg/m^{3.}

A porous polymer as described herein may have any suitable refractive index. For example, the porous polymer may have a refractive index in air of between about 1.28 and about 1.45. In some embodiments, the porous polymer has a refractive index equal to or less than about 1.45, such as from about 1.2 to about 1.45 including all intermediate values and ranges, such as 1.2 to 1.4, 1.2 to 1.35, 1.25 to 1.4, 1.3 to 1.45, 1.3 to 1.4, 1.35 to 1.45, 1.35 to 1.4, and like values and ranges. If desired, the porous articles can be made to be nearly transparent when immersed in culture media by matching the article's refractive index with or in close proximity to that of the media. A nearly transparent article enables, for example, deeper penetration for optical imaging of the cells residing inside the article. The imaging penetration of two-photon fluorescence microscopy in porous substrates made of polydimethylsiloxane (PDMS) can reach, for example, from about 100 to about 1,000 microns, and deeper than about 500 microns compared to, for example, only about 90 microns in a polyvinylalcohol (PVA) based porous article.

The refractive index of a typical aqueous cell culture media can be, for example, from about 1.33 to about 1.37. In embodiments, the refractive index of porous polymer and the refractive index of a typical aqueous cell culture media are selected so that there is a match or near match of the respective refractive indices, for example, where the difference in the respective refractive indices is less than about ±0.5, preferably less than about ±0.15, more preferably less than about ±0.12, and even more preferably less than about ±0.10.

The porous polymer article can have an optical density of, for example, from about 0 to about 1, and an optical penetration depth of, for example, from about 100 to about 1,000 microns or more. The porous polymer article can comprise a polymer, copolymer, or like material, having a molecular weight of from about 500 to about 500,000 Daltons.

3. Cell Culture Article

A porous polymer substrate may be associated with a cell culture article in any suitable manner. For example, polymerization of a mixture for forming the porous substrate can be accomplished on cell culture article substrate. Additionally or alternatively, polymerizing the mixture can be accomplished as, for example, a pre-form, which is a molded form in a variety of useful shapes, and optionally attached to or associated with, for example, a substrate, a vessel, or like supports, i.e., polymerizing a mixture comprising at least one monomer and at least one pore-former particulate material on a substrate to form a continuous polymer matrix and a discontinuous particulate phase on the substrate. If desired, a tie-layer or conversion coating, such as an aminosilane can be selected to enhance adhesion of the porous polymer to a substrate such as glass.

Any suitable cell culture article may include a porous polymer substrate. Examples of suitable cell culture articles with which a porous polymer substrate may be associated include single and multi-well plates, such as 6, 12, 96, 384, and 1536 well plates, jars, petri dishes, flasks, beakers, plates, roller bottles, slides, such as chambered and multichambered culture slides, tubes, cover slips, membranes, hollow fibers, beads and microcarriers, cups, spinner bottles, perfusion chambers, bioreactors, CellSTACK® and fermenters.

Such articles may be made of any suitable material, including a metal, such as a metal oxide; a ceramic substance; a glass, a plastic, a polymer or co-polymer, any combinations thereof, or a coating of one material on another.

In some embodiment, e.g., where the cell culture article is a microcarrier, the article may consist of or consist essentially of the porous polymer substrate.

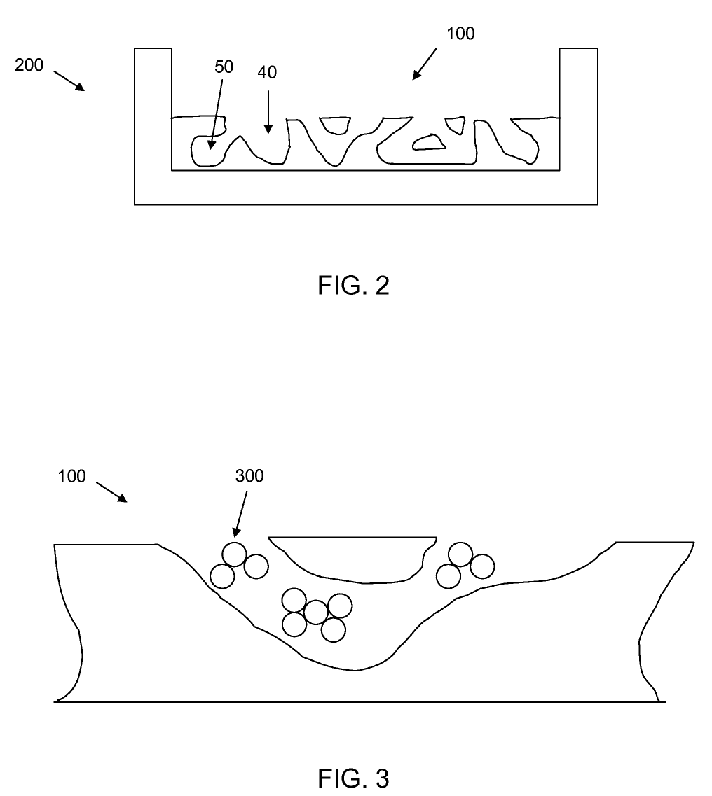

Referring now to FIG. 2, a schematic cross-section of a cell culture article 200 including a porous substrate 100 is shown. The porous polymer cell culture substrate 100 is disposed adjacent a surface the underlying article 200, such as the bottom surface of a well. All or part of the surface or surfaces of the article 200 that may come into contact with cells or cell culture medium may be covered with the porous culture substrate 100. Representative pores 40 and interstices 50 are identified in the embodiment depicted in FIG. 2.

As shown in the schematic cross-section of FIG. 3, the pores and interstices of the porous substrate 100 are sufficiently large to allow cells to form clusters 300 with the pores and interstices.

4. Culturing cells

A cell culture article having a porous polymer substrate as described above may be seeded with cells. The cells may be of any cell type. For example, the cells may be connective tissue cells, epithelial cells, endothelial cells, hepatocytes, skeletal or smooth muscle cells, heart muscle cells, intestinal cells, kidney cells, or cells from other organs, stem cells, islet cells, blood vessel cells, lymphocytes, cancer cells, or the like. The cells may be mammalian cells, preferably human cells, but may also be non-mammalian cells such as bacterial, yeast, or plant cells. In numerous embodiments, the cells are mammary epithelial cells. As used herein, mammary epithelial cells include primary cells, immortalized cell lines, and breast cancer cells having an epithelial origin. Breast cancer cells can be invasive or non-invasive.

Prior to seeding cells, the cells may be harvested and suspended in a suitable medium, such as a growth medium in which the cells are to be cultured once seeded onto the surface. For example, the cells may be suspended in and cultured in a serum-containing medium, a conditioned medium, or a chemically-defined medium. One or more growth or other factors may be added to the medium as desired.

The cells may be seeded at any suitable concentration. Typically, the cells are seeded at about 10,000 cells/cm² of substrate to about 500,000 cells/cm². For example, cells may be seeded at about 50,000 cells/cm² of substrate to about 150,000 cells/cm². However, higher and lower concentrations may readily be used. The incubation time and conditions, such as temperature, CO₂ and O₂ levels, growth medium, and the like, will depend on the nature of the cells being cultured and can be readily modified. The amount of time that the cells are incubated on the surface may vary depending on the cell response desired.

Embodiments of cell culture articles having a porous substrate as described herein are capable of supporting culture of mammary epithelial cells, where such cells exhibit in vivo-like morphology or characteristics, such as formation of acini structures in non-malignant mammary epithelial cells, formation of mass cell structures with robust cell-cell interaction and disorganized nuclei in non-invasive breast cancer cells, formation of elongated cell bodies resembling invasive processes in invasive malignant breast cancer cells, response to anti-cancer agents by breast cancer cells, or reversion of malignant phenotype of breast cancer cells. Acinus structures are clusters of cells that resemble a many-lobed berry, such as a raspberry. In vivo, mammary epithelial cells that form acini form the tissue of the breast gland that produce fluid or milk.

5. Screening Anti-Cancer Candidate Compounds

The cultured cells may be used for any suitable purpose. For example, the cells may be used to determine whether agents, such as anti-cancer agents, have desirable effects on the cultured cells. Because the cell culture articles described herein can support the culture and in-vivo like morphology and characteristics of mammary epithelial cells, including breast cancer cells, the articles may be advantageously used to test the ability of candidate anti-cancer agents have desirable effects on breast cancer cells.

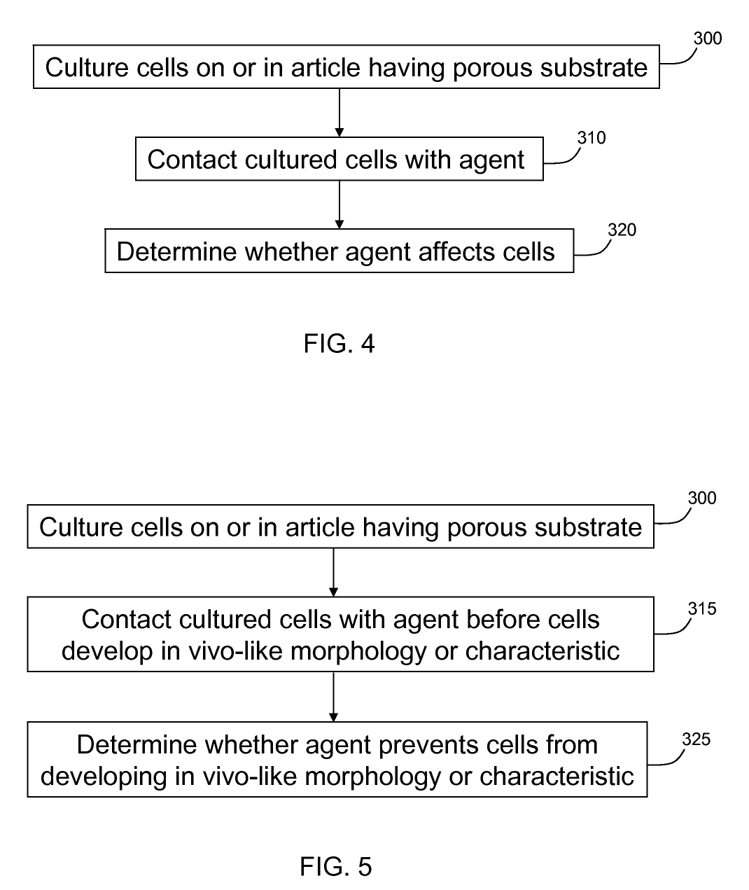

Some representative methods for screening compounds are shown in the flow diagrams of FIGS. 4-7. As shown in FIG. 4, cells, such as mammary epithelial cells, may be cultured on or in a cell culture article having a porous substrate (300) as discussed above. The cultured cells may be contacted with a candidate agent (310) and the effects of the agent on the cells may be determined (320).

In the method depicted in FIG. 5, cells, such as malignant mammary epithelial cells, may be cultured on or in a cell culture article having a porous substrate (300). The cultured cells may be contacted with a candidate compound before cells develop an in vivo-like morphology or characteristic (315), such as mass cell structures with robust cell-cell interaction and disorganized nuclei in non-invasive breast cancer cells, formation of elongated cell bodies resembling invasive processes in invasive malignant breast cancer cells, or cellular proliferation for invasive or non-invasive breast cancer cells. A determination may then be made as to whether the agent or candidate compound prevented the cells from developing the in vivo-like morphology or characteristic (325), e.g., by comparing to cells culture in the absence of the agent or candidate.

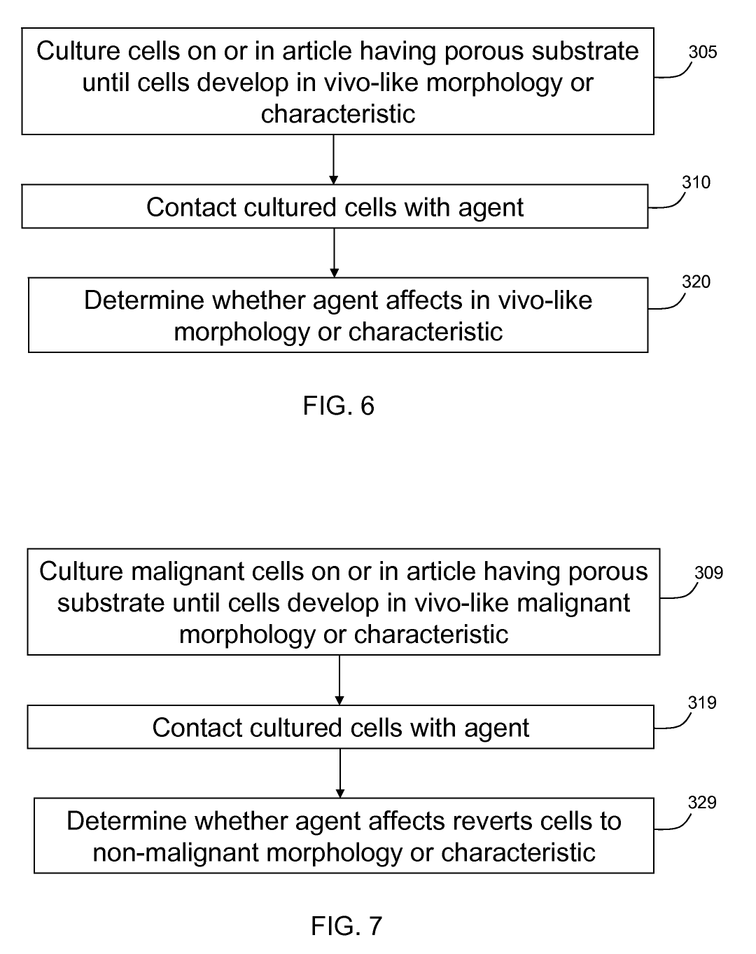

In the method depicted in FIG. 6, cells, such as malignant mammary epithelial cells, may be cultured on or in an a cell culture article having a porous substrate for a sufficient amount of time until an in vivo-like morphology or characteristic has been developed (305). Examples of in vivo-like morphologies or characteristics that malignant mammary epithelial cells may exhibit include mass cell structures with robust cell-cell interaction and disorganized nuclei in non-invasive breast cancer cells, formation of elongated cell bodies resembling invasive processes in invasive malignant breast cancer cells, or cellular proliferation for invasive or non-invasive breast cancer cells. The in vivo-like cultured cells may then be contacted with the agent or candidate compound (310), e.g. via introduction into the cell culture medium, and a determination may be made as to whether the agent or compound affects the vivo-like morphology or characteristic of the cells (320). For example, it can be determined whether the cell number has been reduced, whether the invasive processes have diminished, or the like.

In the method depicted in FIG. 7, malignant cells, such as malignant mammary epithelial cells, may be cultured on or in an a cell culture article having a porous substrate for a sufficient amount of time for an in vivo-like malignant morphology or characteristic develops (309). The cells are then contacted with the agent or candidate compound (319) and a determination is made as to whether the agent or candidate reverts the cells to a non-malignant morphology or characteristic (329), such as formation of an acinus structure. It may be desirable to culture the malignant cells on a substrate that encourages development of in-vivo like morphologies or characteristics of non-malignant cells in such methods. For example, if a cell culture substrate that encourages formation of acinus structures in non-malignant cells, such as a hydrophobic porous substrate, is used, reversion to the in vivo-like morphology may be observed. However, if the cell culture substrate is not capable of supporting in-vivo like morphology or characteristics of non-malignant cells, such the ability of the agents to revert malignant cells to non-malignant morphologies or characteristics may not be detectable.

It will be understood that these are just some examples of methods for using the cell culture apparatuses described herein, and that other uses will be evident to those of skill in the art. In addition, while much of the discussion provide herein relates to culture of mammary epithelial cells, the articles described herein may be used for culturing any cells.

In the following, non-limiting examples are presented, which describe various non-limiting embodiments of the cell culture articles, porous substrates, and methods discussed above.

### EXAMPLES

### Example 1

### Fabrication of Porous Substrate

Porous polydimethylsiloxane (PDMS) substrates were generated by mixing a PDMS pre-polymer and a curing agent using the Sylgard 182 kit available from Dow Corning in a ratio of 10 to 1. The mixture was closely packed with sugar crystals of size ranging from 212-250 micrometers, which was then filled in a mold and cured at 100 degrees for one hour. The sugar in the cured polymer was washed out in an ultrasonic bath, forming porous PDMS substrates. The resulting porous PDMS was released from the mold and assembled into multi-well plate for cell culture.

Depending on the density of wells in the plates, the plates were prepared as follows: (1) for 96-well plate, porous PDMS was molded in a 96-well format on a piece of glass insert, then the insert was released from the mold and glued to the bottom of 96-well holy plate (plate without the bottom) by double-sided pressure sensitive adhesive (PSA) plate; or (2) for plates of density lower than 96 wells, such as 48, 24, 12, etc, porous PDMS was molded on an plastic or metal plate in corresponding format, then individual disc of porous PDMS were released and placed on the bottom of individual well of a well plate. To prevent the porous PDMS floating during cell culture, the PDMS substrate may be glued to the bottom of the plate by using PDMS pre-polymer or may be directly bonded to an oxygen plasma treated plate surface.

### Example 2

### Culture of Non-malignant Breast Cells on Porous Substrate

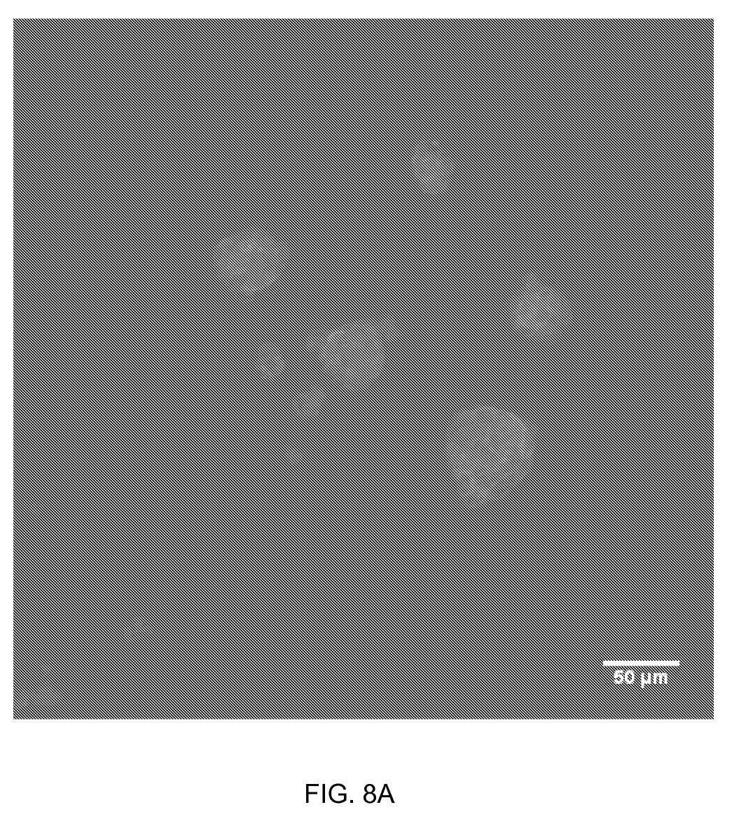

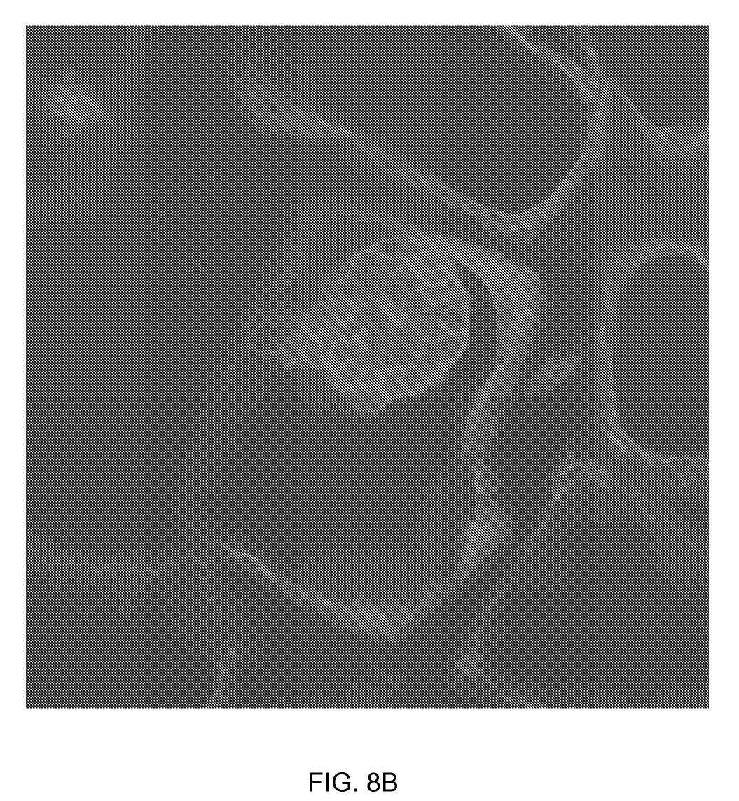

Non-malignant breast cells MCF-10A were seeded in wells having the porous PDMS substrates described in Example 1. 10,000 cells were seeded and cultured in MEBM/F12 cell culture medium with 5% horse serum, 5% pen/strep, 20 ng/ml of hEGF, 0.5 μml of hydrocortisone, 100 ng/ml of Cholera toxin and 10 μg/ml of insulin. The medium was changed on alternate days. Following two week of culture, the cells were stained with Rhodamine phalloidin (F-actin staining) and imaged with confocal fluorescence microscope. The confocal image of MCF-10A, as shown in FIGS. 8A-B (with FIG. 8B being of higher magnification), reveals that MCF-10A formed a structure of organized nuclei and robust cell-cell adhesion. The observed cell morphology closely resembled the in-vivo acini structure of non-malignant mammary epithelial cells. This is in contrast to MCF-10A cells cultured on traditional 2D surfaces, on which the cells form monolayer.

### Example 3

### Culture of Malignant Breast Cells on Porous Substrate

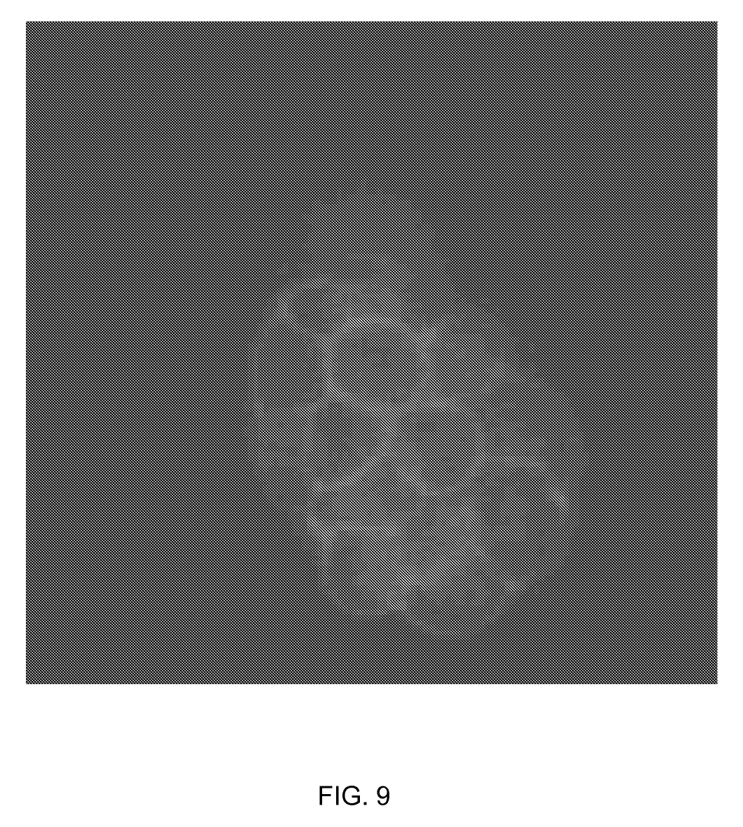

Malignant breast cells MCF-7 were seeded in wells having the porous PDMS substrates described in Example 1. 10,000 cells were seeded and cultured in EMEM cell culture medium with 10% Fetal Bovine Serum, 5% pen/strep, and 10 μg/ml of insulin. The medium was changed on alternate days. Following two weeks of culture, the cells were stained with Rhodamine phalloidin (F-actin staining) and DAPI nuclei counter stain and imaged with confocal fluorescence microscope as shown in FIG. 9. The image shows that MCF-7 cells formed a mass structure of disorganized nuclei but maintain robust cell-cell adhesion.

### Example 4

### Culture of Invasive Breast Cancer Cells on Porous Substrate

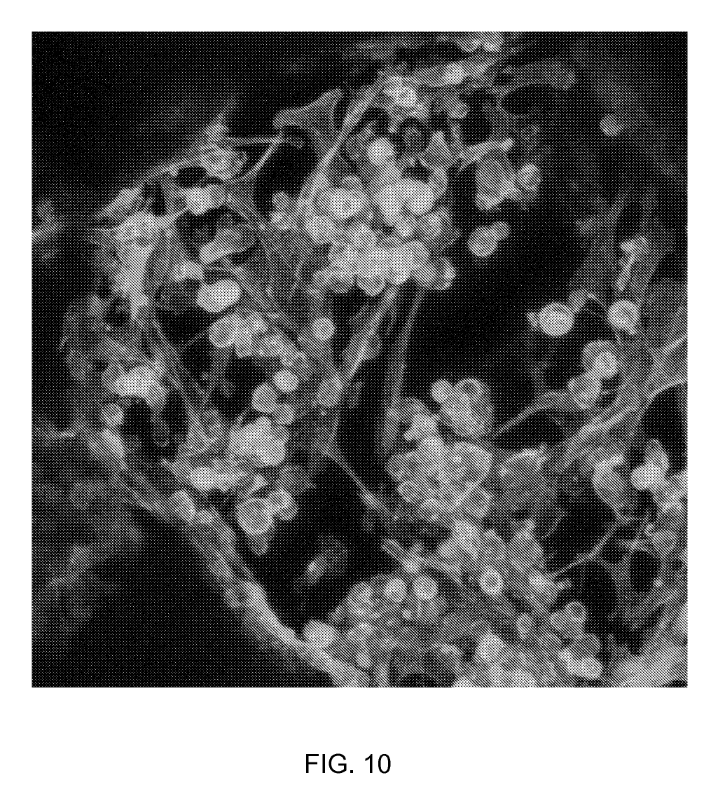

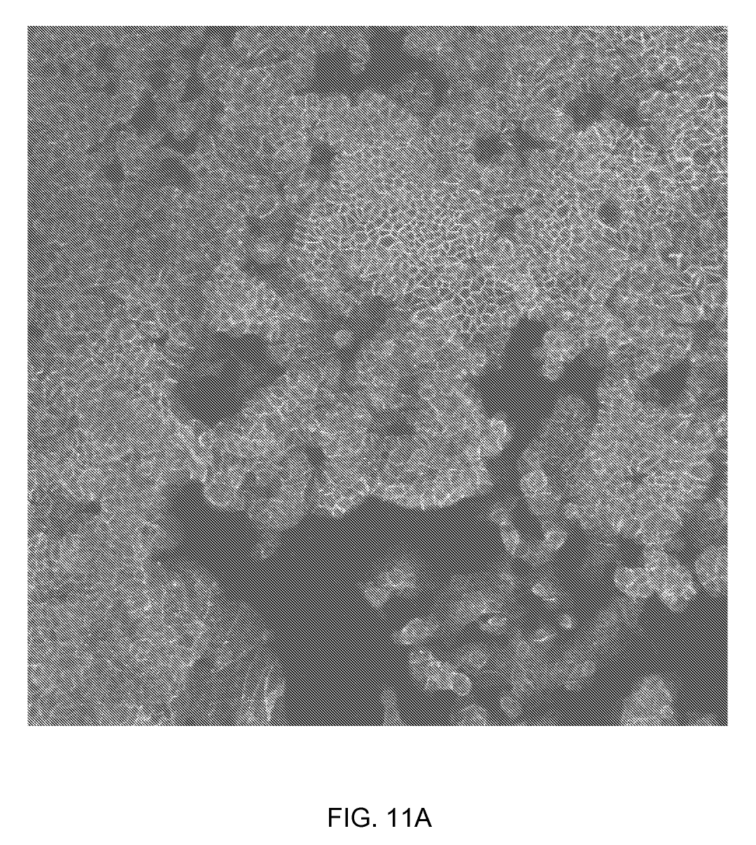

Invasive breast cells MDA-MB-231 were seeded in wells having the porous PDMS substrates described in Example 1. 10,000 cells were seeded and cultured in Leibovitz L-15 cell culture medium with 10% Fetal Bovine Serum and 5% pen/strep. The medium was changed on alternate days. Following two weeks of culture, the cells were stained with Rhodamine phalloidin (F-actin stain) and DAPI nuclei counter stain and imaged with confocal fluorescence microscope as shown in FIG. 10. The fluorescence image shows that MDA-MB-231 cells have formed a structure of disorganized nuclei and elongated cell body with invasive processes. As with the non-malignant cells in Example 2 and the malignant cells in Example 3, the invasive MDA-MB-231 cells cultured in porous PDMS closely resemble those reported in Matrigel culture, but vary significantly from conventional 2D cell culture.

### Example 5

### Effects of Porous Substrate on Cellular Function

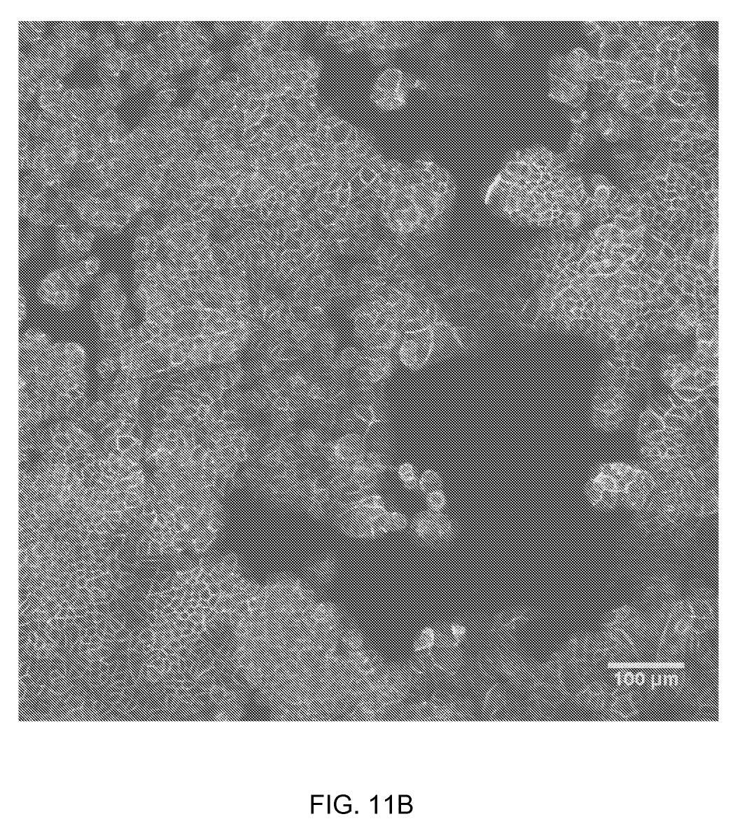

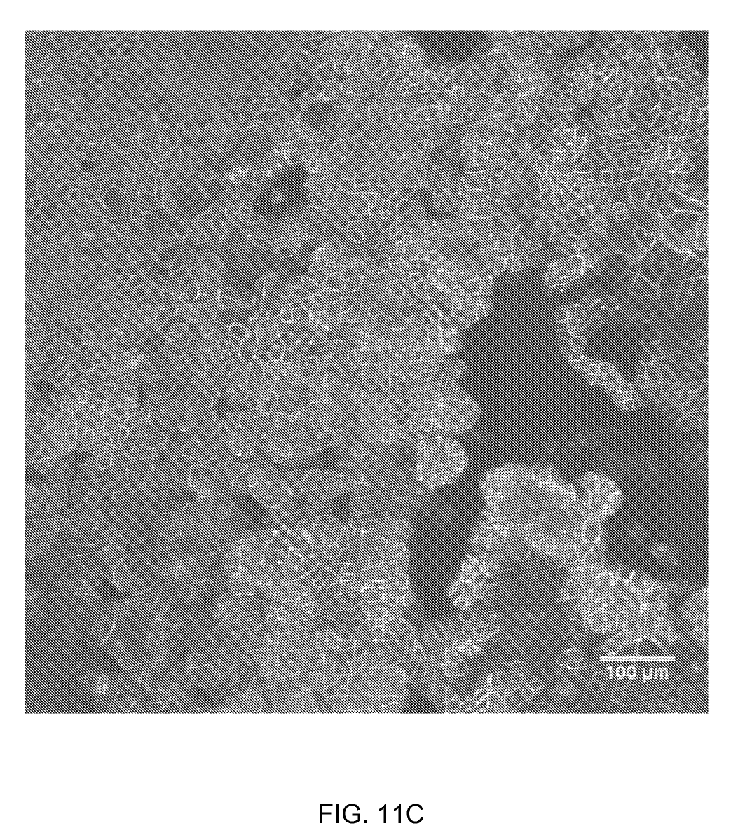

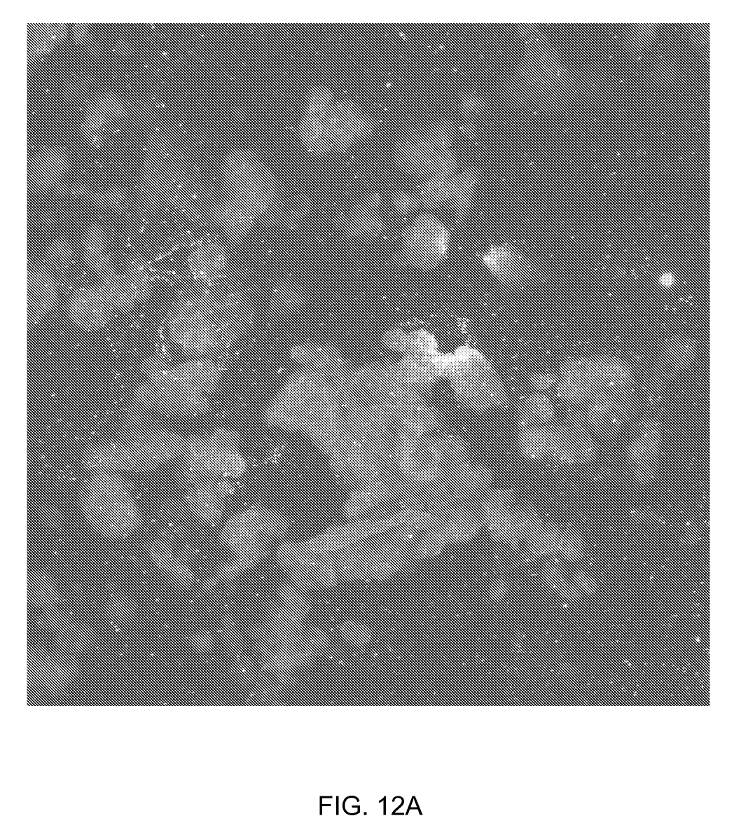

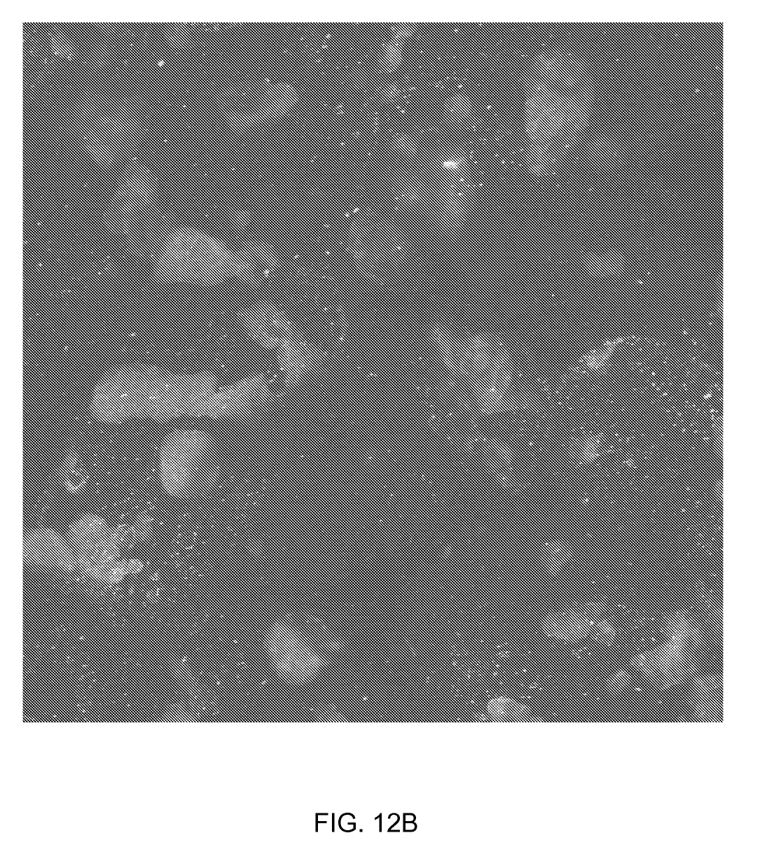

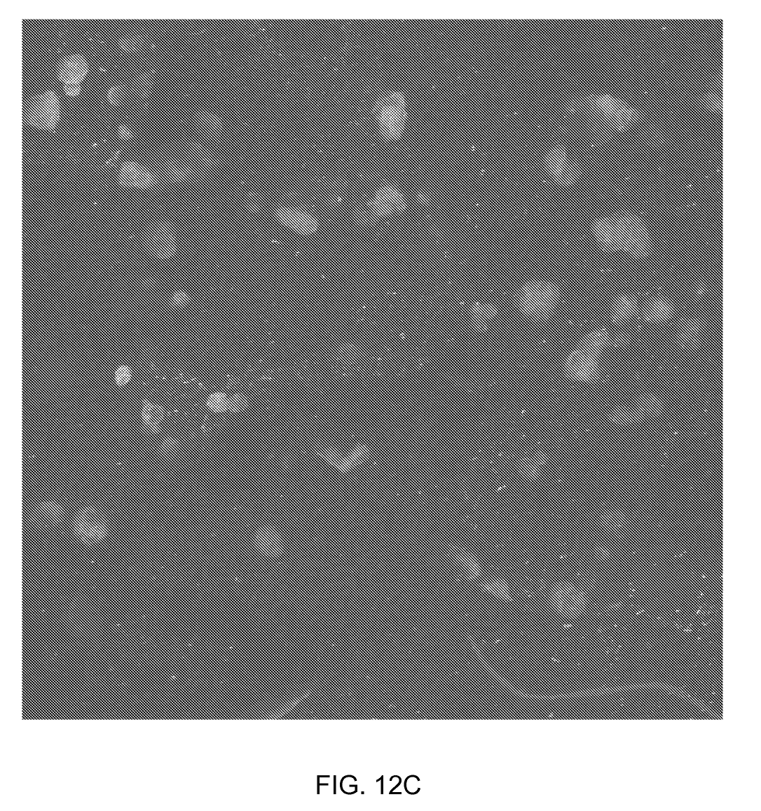

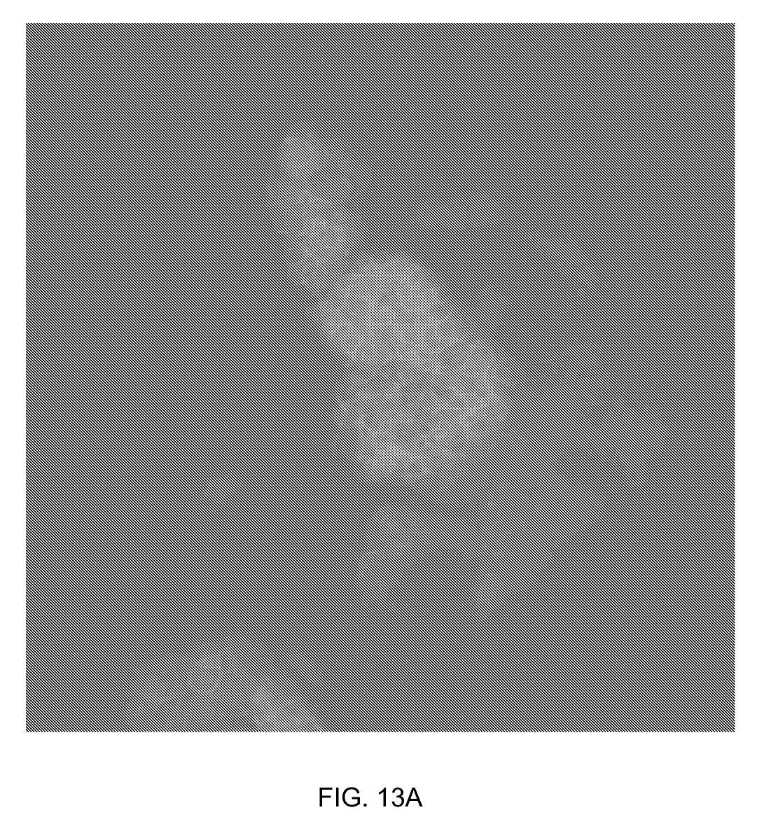

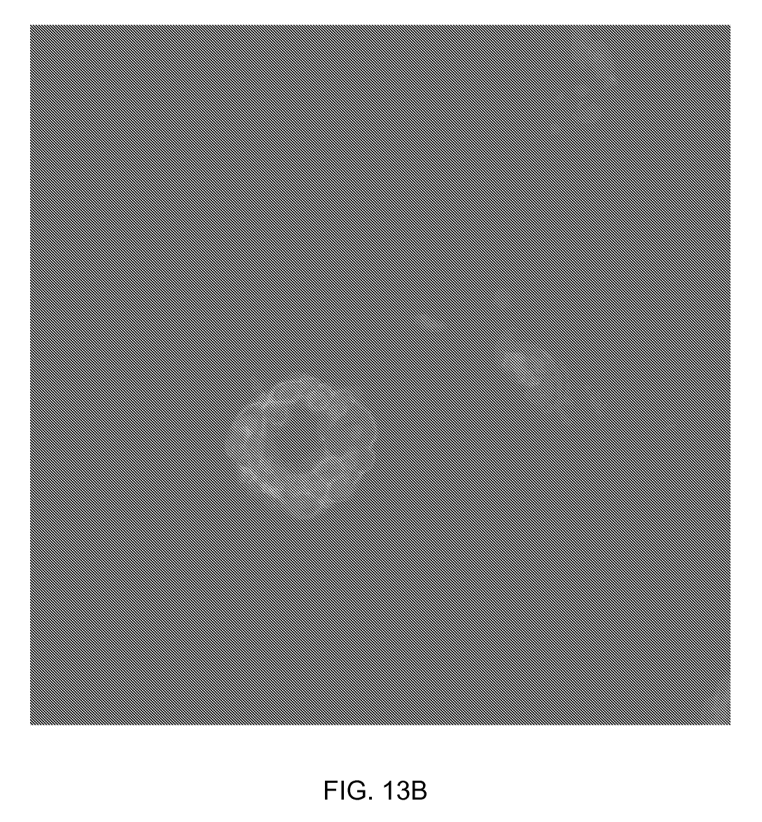

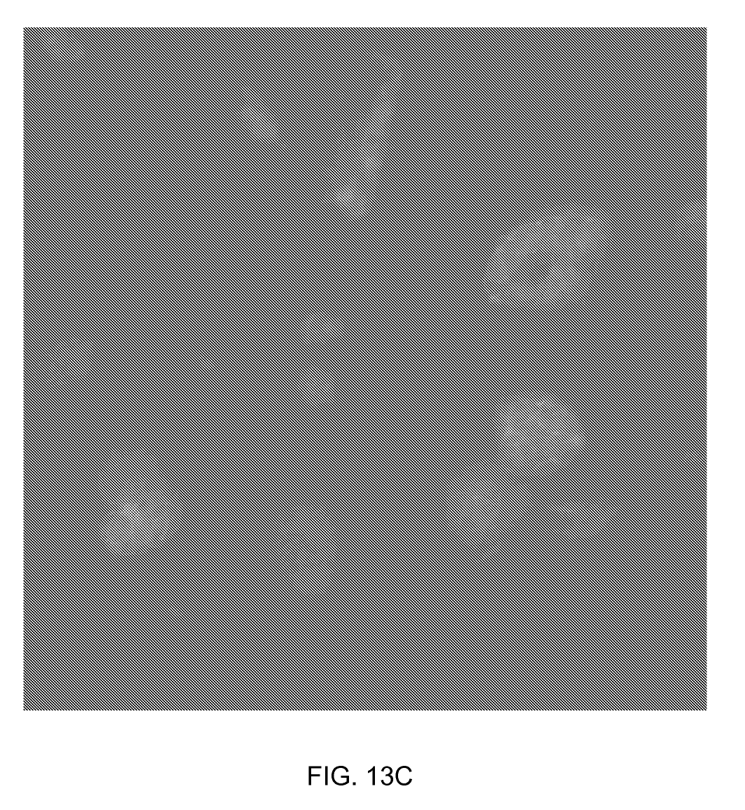

Differences in cellular morphology often contribute to differences in cellular function. To understand the difference of cellular functions between breast cells cultured in a porous substrate and on a 2D culture surface, 10,000 MCF-7 malignant cell were seeded and cultured in porous PDMS (as described in Example 1) and a TCT culture surface (tissue culture treated polystyrene, “TCT”, Coming Inc) under the same conditions. The cells were treated with 4 μM of PD98059 (MAPK inhibitor) and 4 μM of LY294002 (PI3K inhibitor) on alternate days for 15 days. Both PD98059 and LY294002 have been reported to reverse malignant behaviors of breast cancer cells to certain degrees in Matrigel 3D culture system. As shown in FIGS. 11A-C both treatments failed to induce noticeable difference for the cells cultured on TCT surface. However, both agents significantly reduced the colony size (FIGS. 12A-C) and reversed malignant phenotype from mass structure to normal acini structure among the cells cultured in porous PDMS substrates (FIGS. 13A-C).

In FIGS. 11A-C, the cells were stained for E-cadherin and counterstained with a DAPI nuclei stain. In FIG. 11A, untreated cells are shown at 14 days of culture. In FIG. 11B, cells treated with PD98059 are shown at 14 days of culture. In FIG. 11C, cells treated with LY294002 are shown at 14 days of culture. The green (E-cadherin) and blue (nuclei) stains cannot be seen in the black and white reproductions presented herein. But, it is apparent from these images that no reversion of malignant phenotype or other substantial change was observed in the drug-treated cells cultured on the 2D substrate.

In FIGS. 12A-C, the cells were stained with Rhodamine phalloidin (F-actin staining) and counterstained with a DAPI nuclei stain. In FIG. 12A, untreated cells are shown at 14 days of culture. In FIG. 12B, cells treated with PD98059 are shown at 14 days of culture. In FIG. 12C, cells treated with LY294002 are shown at 14 days of culture. The red (F-actin) and blue (nuclei) stains cannot be seen in the black and white reproductions presented herein. But, it is apparent from these images that both agents significantly reduced the colony size.

In FIGS. 13A-C, the cells were stained with Rhodamine phalloidin (F-actin staining) and counterstained with a DAPI nuclei stain. In FIG. 13A, untreated cells are shown at 14 days of culture. In FIG. 13B, cells treated with PD98059 are shown at 14 days of culture. In FIG. 13C, cells treated with LY294002 are shown at 14 days of culture. The red (F-actin) and blue (nuclei) stains cannot be seen in the black and white reproductions presented herein. But, it is apparent from these images that both agents reversed malignant phenotype from mass structure to normal acini structure.

### Example 6

### Comparison of Cells Cultured on Porous Substrate and 2D Substrate

Porous PDMS substrates having different pore sizes were also generated as described above in Example 1, but with different sized sugar crystals. The sugar crystal sized used to generate the porous PDMS ranged from about 200 micrometers to about 800 micrometers. The porous PDMS substrates were assembled into multi-well plates and malignant and non-malignant breast cells were cultured on the porous substrates, generally as described above with regard to Examples 2 and 3. The varying pore size did not appear to affect the morphology of the cells (data not shown).

Thus, embodiments of SUBSTRATE AND METHOD FOR CULTURING BREAST CELLS are disclosed. One skilled in the art will appreciate that the cell culture articles, porous substrates, kits and methods described herein can be practiced with embodiments other than those disclosed. The disclosed embodiments are presented for purposes of illustration and not limitation.

What is claimed is:

1. A cell culture article for culturing mammary epithelial cells, comprising:
a porous substrate having a plurality of pores and a plurality of interstices in communication with the pores,
wherein the pores and interstices are formed from voids generated by pore-former particles having an average particle size of between 75 micrometers and 1000 micrometers,
wherein the substrate has a contact angle of greater than 30 degrees, and
wherein the article is free of components of unknown composition.

2. The cell culture article of claim 1, wherein the porous substrate is formed from polydimethylsiloxane.

3. The cell culture article of claim 1, wherein the porous substrate consists essentially of polydimethylsiloxane.

4. The cull culture article of claim 1, further comprising a well having a bottom surface, wherein the porous substrate is adjacent the bottom surface of the well.

5. The cell culture article of claim 1, wherein the pores and interstices are formed from voids generated by pore-former particles having an average particle size of between 200 micrometers and 250 micrometers.

6. The cell culture article of claim 1, wherein the substrate has a contact angle of between 80 degrees and 130 degrees.

7. A method, comprising:
seeding mammary epithelial cells in or on a cell culture article that includes a porous substrate having a plurality of pores and a plurality of interstices in communication with the pores, wherein the pores and interstices are formed from voids generated by pore-former particles having an average particle size of between 75 micrometers and 1000 micrometers; and
contacting the seeded cells with a cell culture medium.

8. The method of claim 7, wherein the mammary epithelial cells comprise non-malignant mammary epithelial cells, and wherein the method further comprises culturing the cells in or on the article for a sufficient time for the cells to form an acinus structure in the pores or interstices.

9. The method of claim 8, wherein the non-malignant mammary epithelial cells comprise primary cells.

10. The method of claim 7, wherein the mammary epithelial cells comprise malignant mammary epithelial cells.

11. The method of claim 10, wherein the malignant mammary epithelial cells comprise non-invasive malignant mammary epithelial cells, and wherein the method further comprising culturing the non-invasive malignant mammary epithelial cells on or in the article for a sufficient time for the non-invasive malignant mammary epithelial cells to form clusters in the pores or interstices.

12. The method of claim 10, wherein the malignant mammary epithelial cells comprise invasive malignant mammary epithelial cells, and wherein the method further comprises culturing the invasive malignant mammary epithelial cells on or in the article for a sufficient time for the invasive malignant mammary epithelial cells to form elongate cell bodies resembling invasive processes.

13. The method of claim 7, wherein the pores and interstices are formed from voids generated by pore-former particles having an average particle size of between 75 micrometers and 1000 micrometers.

14. The method of claim 7, wherein the pores and interstices are formed from voids generated by pore-former particles having an average particle size of between 200 micrometers and 250 micrometers.

15. The method of claim 7, wherein the article is free of components of unknown composition.

16. The method of claim 7, wherein the cells do not attach to the substrate surface.

17. The method of claim 7, wherein the substrate has a contact angle of 30 degrees or more.

18. The method of claim 7, wherein the substrate has a contact angle of between 80 degrees and 130 degrees.

19. The method of claim 7, wherein the substrate is formed from polydimethylsiloxane.

20. The method of claim 7, wherein the substrate consists essentially of polydimethylsiloxane.

21. A method for screening agents that may be beneficial for treating breast cancer, comprising:
seeding malignant mammary epithelial cells in or on a cell culture article that includes a porous substrate having a plurality of pores and a plurality of interstices in communication with the pores, wherein the pores and interstices are formed from voids generated by pore-former particles having an average particle size of between 75 micrometers and 1000 micrometers;
culturing the malignant mammary epithelial cells in or on the article for a sufficient time for the malignant mammary epithelial cells to develop an in vivo-like characteristic of malignant mammary epithelial cells; and
contacting the cultured cells with the agent before or after the time sufficient for the malignant mammary epithelial cells to develop the in vivo-like characteristic malignant mammary epithelial cells.

22. The method of claim 21, further comprising determining whether the agent effects cell viability or number or the development or maintenance of the in vivo-like characteristic of malignant mammary epithelial cells.

23. The method of claim 21, wherein the malignant mammary epithelial cells are invasive cells, and wherein the in vivo-like characteristic is elongate cell bodies resembling invasive processes.

24. The method of claim 21, further comprising determining whether contacting the cells with the agent results in the cells forming an acinus structure.

25. The method of claim 21, wherein the pores and interstices are formed from voids generated by pore-former particles having an average particle size of between 200 micrometers and 250 micrometers.

26. The method of claim 21, wherein the substrate has a contact angle of greater than 30 degrees.

27. The method of claim 21, wherein the substrate has a contact angle of between 80 degrees and 120 degrees.
